SEQUENCE LISTING (2 sequences)

SEQ ID NO: 1 - protein, 1048 aa, synthetic construct
AIKEMPQPKTFGELKNLPLLNTDKPVQALMKIADELGEIFKFEAPGWVTRYLSSQRLIKEACDESRFDKNLWQALKYLRDILGDGLGSSWTHEKNWKKAHNILLPSFSQQAMKGYHAMMVDIAVQLVQKWERLNADEHIEVPEDMTRLTLDTIGLCGFNYRFNSFYRDQPHPFITSTVRALDEAQNKQQRANPDDPAYDENKRQFQEDTKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTGPWFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 2 - DNA, 3147 bp, synthetic construct
  source 1..3147: mol_type = other DNA
gcaattaaagaaatgcctcagccaaaaacgtttggagagcttaaaaatttaccgttattaaacacagataaaccggttcaagctttgatgaaaattgcggatgaattaggagaaatctttaaattcgaggcgcctggttgggtaacgcgctacttatcaagtcagcgtctaattaaagaagcatgcgatgaatcacgctttgataaaaacttatggcaagcgcttaaatacctaagagacatcctaggagacgggttaggatcaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtacggtccgtgcactggatgaagcacaaaacaagcaacagcgagcaaatccagacgacccagcttatgatgaaaacaagcgccagttccaagaagatacaaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactggtccttggttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

(19) United States
(12) Patent Application Publication
(10) Pub. No.: US 2025/0327109 A1
(43) Pub. Date: Oct. 23, 2025

(54) CHEMOENZYMATIC METHOD FOR SYNTHESIZING STEROID HE3286

(71) Applicant: Hubei University, Wuhan (CN)

(72) Inventors: Aitao Li, Wuhan (CN); Zili Zhang, Wuhan (CN); Qian Li, Wuhan (CN); Chenghua Gao, Wuhan (CN)

(21) Appl. No.: 19/218,238

(22) Filed: May 24, 2025

Related U.S. Application Data
(63) Continuation of application No. PCT/CN2024/107717, filed on Jul. 26, 2024.

(30) Foreign Application Priority Data
Apr. 19, 2024 (CN) 2024104747629

(51) Int. Cl. C12P 33/06; C07J 1/00

(52) U.S. Cl. CPC C12P 33/06; C07J 1/0048

(57) ABSTRACT

A chemoenzymatic method for synthesizing steroid HE3286 is provided, comprising: screening cytochrome P450 mutant LG-23 capable of catalyzing 7β-hydroxylation of dehydroepiandrosterone; enzymatically converting dehydroepiandrosterone to 7β-hydroxy-dehydroepiandrosterone using the P450 BM3 mutant enzyme; and chemically performing alkynylation at the C17th position carbonyl group to generate steroid HE3286. The steroid HE3286 synthesis method not only features simplified synthetic steps and high catalytic selectivity, but also offers mild reaction conditions, low cost, and environmentally friendly efficiency. This approach holds significant application value for advancing the development of steroid pharmaceuticals.

### FIELD OF THE DISCLOSURE

The present disclosure relates to a chemoenzymatic method for synthesizing steroid HE3286.

### STATEMENT REGARDING SEQUENCE LISTING

The sequence listing associated with this application is provided in text format in lieu of a paper copy and is hereby incorporated by reference into the specification. The name of the XML file containing the sequence listing is ZL251463-USP1.xml. The XML file is 7,136 bytes; is created on Apr. 28, 2025; and is being submitted electronically via patent center.

### BACKGROUND

Currently, there are over 400 types of steroid drugs, making them the second-largest family of drugs after antibiotics. Steroid drugs are widely used in the clinical treatment of various diseases, including autoimmune disorders, inflammation, cancer, coronavirus infections, osteoporosis, and more. The introduction of different functional groups (such as hydroxyl groups) into the steroid backbone is crucial for modulating the physiological and pharmacological activities of these drugs.

Steroid HE3286 (CAS: 1001100-69-1), chemically named as 17α-ethynyl-androst-5-ene-3β,7β, 17β-triol, is indicated for the prevention and treatment of metabolic disorders including type 2 diabetes and hyperglycemia, as well as autoimmune diseases such as rheumatoid arthritis.

Patent WO 2009149392 describes three synthetic routes for steroid HE3286. The first synthetic route employs dehydroepiandrosterone (DHEA) as the starting material and proceeds through the following steps: (i) protection of the 3-hydroxyl group with TMSCI, (ii) acetylation of the 17-carbonyl group, (iii) protection of the 3rd position with an acetyl group, (iv) oxidation to afford the 7-keto compound, (v) reduction to yield the 7β-hydroxy derivative, (vi) hydrolysis at the 3rd position to give the target compound HE3286. This six-step synthesis achieves an overall yield of 15%.

The second synthetic route employs dehydroepiandrosterone acetate as the starting material and proceeds through the following sequence: (i) acetal protection at the 17-position, (ii) sequential oxidation and reduction at the 7th position to obtain the 7β-hydroxy intermediate, (iii) deprotection of the 17th-acetal group, (iv) hydrolysis at the 3rd position, (v) TMS protection of both 3rd and 7-hydroxyl groups, (vi) ethynylation at the 17th position, (vii) final TMS deprotection to yield the target compound HE3286. This eight-step synthesis process achieves an overall yield of 6%.

The third synthetic route also employs dehydroisoandrosterone acetate as the starting material and proceeds through the following sequence: (i) oxidation at the 7th position, (ii) hydroxylation at the 17-position, (iii) reduction at the 7th position, (iv) hydrolysis at the 17th position to yield 7β-hydroxy dehydroepiandrosterone acetate intermediate, (v) hydrolysis at the 3rd position, (vi) TMS protection of both 3rd and 7th hydroxyl groups, (vii) ethynylation at the 17th position, (viii) final TMS deprotection to obtain the target compound HE3286. This eight-step synthetic route achieves an overall yield of 30%.

In Patent CN114478672A, the target compound HE3286 was synthesized from 3β, 7α, 15α-trihydroxyandrost-5-en-17-one (CAS: 2963-69-1) through a series of reactions including rearrangement, diesterification, elimination, hydrogenation, ethynylation, and hydrolysis, achieving an overall yield of 80%.

In Patent WO 2009149392, the synthesis of steroid HE3286 not only involved lengthy routes but also resulted in low overall yield. Although Patent CN114478672A significantly improved the overall yield of HE3286, the process still suffers from issues such as prolonged reaction pathways, cumbersome operations, and high synthesis costs, making it unsuitable for large-scale industrial production.

### SUMMARY

To address the various challenges in steroid HE3286 synthesis, the present disclosure provides a chemoenzymatic route. Specifically: first, dehydroepiandrosterone undergoes C7β-hydroxylation via enzymatic catalysis to yield 7β-hydroxy-dehydroepiandrosterone, followed by chemical ethynylation to obtain steroid HE3286.

A chemoenzymatic method for synthesizing steroid HE3286, comprising the following steps:
(1) converting dehydroepiandrosterone into 7β-hydroxy-dehydroepiandrosterone under the action of 7β-hydroxylase;
(2) alkynylating the carbonyl group at the C17th position of 7β-hydroxy-dehydroepiandrosterone to obtain steroid HE3286.

Preferably, the 7β-hydroxylase is a cytochrome P450 enzyme, specifically a P450 BM3 mutant. For example, in some embodiments, the 7β-hydroxylase is the P450 BM3 mutant LG-23, whose amino acid sequence is shown in SEQ ID NO:1 and nucleotide sequence is shown in SEQ ID NO:2.

Preferably, step (1) specifically includes: 11) reacting mutant LG-23 with isopropanol dehydrogenase or glucose dehydrogenase, dehydroepiandrosterone, NADP+ cofactor, and isopropanol or glucose;
12) extracting the reaction mixture with ethyl acetate;
13) drying the ethyl acetate extract over anhydrous Na₂SO₄, followed by filtration and concentration under reduced pressure to yield crude 7β-hydroxy-dehydroepiandrosterone; and 14) purifying the crude product by recrystallization.
Preferably, step (2) is specifically performed by reacting 7β-hydroxy-dehydroepiandrosterone with ethynylmagnesium bromide, acetylene gas, or other ethynyl Grignard reagents in an alkynylation reaction.

In some embodiments, step (2) is specifically performed as follows:

Dissolve 0.5-2 g of 7β-hydroxy-dehydroepiandrosterone in 3.2-12.8 mL of tetrahydrofuran (THF). Under ice-bath cooling and nitrogen protection, add dropwise 70-100 mL of a 0.3-0.5 M solution of ethynylmagnesium bromide in THE (18 eq). Allow the reaction to proceed at 0-40° C. while monitoring the progress by thin-layer chromatography (TLC) (dichloromethane:methanol=15:1) until complete substrate conversion is achieved. Upon reaction completion, quench the mixture with a saturated ammonium chloride solution and extract with an equal volume of ethyl acetate. Dry the resulting organic extract over anhydrous sodium sulfate (Na₂SO₄), filter, and concentrate under reduced pressure to remove the solvent. Add diisopropyl ether to the residue for slurrying, cool to induce crystallization, and isolate the product by suction filtration. After drying, the target compound HE3286 is obtained.

In other embodiments, step (2) is specifically performed as follows:
(21) dissolving 7β-hydroxy-dehydroepiandrosterone in an organic solvent, sequentially adding an activator and tert-butyldimethylsilyl chloride (TBDMSCI), and reacting to obtain a 3,7-hydroxyl-protected compound; and
(22) subjecting the C17th carbonyl group of the 3,7-hydroxyl-protected compound to alkynylation to produce steroid HE3286.

Preferably, the organic solvent in step (21) is selected from THF, acetonitrile (MeCN), dichloromethane (DCM), and N,N-dimethylformamide (DMF), with THE being more preferred.

Preferably, the activator in step (21) is selected from imidazole, pyridine, 4-dimethylaminopyridine (DMAP), 2,6-lutidine, triethylamine, N,N-diisopropylethylamine (DIPEA), and 1,8-diazabicyclo[5.4.0]undec-7-ene (DBU), with imidazole being particularly preferred. In some embodiments, the molar ratio of TBDMSCI to 7β-hydroxy-dehydroepiandrosterone is 3-5:1, the molar ratio of imidazole to TBDMSCI is 1.2-1.5:1, and the reaction temperature in step (21) does not exceed 50° C.

Preferably, step (22) is specifically performed as follows:

The 3,7-hydroxyl-protected compound is mixed with a cosolvent and reacted with ethynylmagnesium bromide, acetylene gas, or another ethynyl Grignard reagent. After the reaction completes, p-toluenesulfonic acid (p-TsOH) is added, followed by concentration under reduced pressure to obtain steroid HE3286.

In preferred embodiments, the cosolvent in step (22) is selected from THF, diethyl ether, isopropyl ether, methyl tert-butyl ether, ethylene glycol dimethyl ether, 2-methyltetrahydrofuran, and 1,4-dioxane.

In preferred embodiments, the ethynyl Grignard reagent in step (22) is ethynylmagnesium bromide, with a molar ratio of ethynylmagnesium bromide to the 3,7-hydroxyl-protected compound ranging from 1.05-30:1.

The present disclosure further provides the application of cytochrome P450 enzymes, vectors/cells expressing cytochrome P450 enzymes, and compositions containing cytochrome P450 enzymes in the production of steroid compounds, wherein the steroid compounds include the steroid HE3286.

Preferably, the cytochrome P450 enzyme is the P450 BM3 mutant LG-23, whose amino acid sequence is shown in SEQ ID NO:1 and nucleotide sequence is shown in SEQ ID NO:2.

The advantages of the technical scheme proposed in the disclosure are:

The present disclosure for the first time discovers that the P450 BM3 mutant possesses catalytic activity for 7β-hydroxylation of dehydroepiandrosterone, and successfully applies it in the synthesis of the steroid HE3286. Specifically, the disclosure is the first to utilize the P450 BM3 mutant to catalyze the one-step conversion of dehydroepiandrosterone into 7β-hydroxy-dehydroepiandrosterone, coupled with isopropanol dehydrogenase for NADPH cofactor regeneration and recycling, followed by chemical alkynylation to produce the steroid HE3286. The method provided by the present disclosure exhibits at least the following advantages: simplified steroid HE3286 synthetic route, significantly improved catalytic selectivity, reduced byproducts with enhanced yield, mild reaction conditions, low cost, and high efficiency with environmental friendliness.

### BRIEF DESCRIPTION OF THE DRAWINGS

Accompanying drawings provide a further understanding of embodiments of the disclosure. The drawings form a part of the disclosure and illustrate the principle of the embodiments of the disclosure along with the literal description. Apparently, the drawings in the description below are merely some embodiments of the disclosure. Any person skilled in the art can obtain other drawings according to these drawings without creative efforts. In the figures:

FIG. 1 shows the synthetic route for preparing steroid HE3286 by combining enzymatic and chemical approaches according to the present disclosure.

FIG. 2 shows the ¹H NMR spectrum (in CDCl₃, 100 MHz) of 7β-hydroxy-dehydroepiandrosterone.

FIG. 3 shows the ¹³C NMR spectrum (in CDCl₃, 400 MHz) of 7β-hydroxy-dehydroepiandrosterone.

FIG. 4 shows the reaction scheme for the C7βhydroxylation of dehydroepiandrosterone catalyzed by P450 BM3 mutant LG-23.

FIG. 5 shows the reaction flowchart for the synthesis of steroid HE3286 in Example 2.

FIG. 6 shows the reaction flowchart for the synthesis of steroid HE3286 in Example 3.

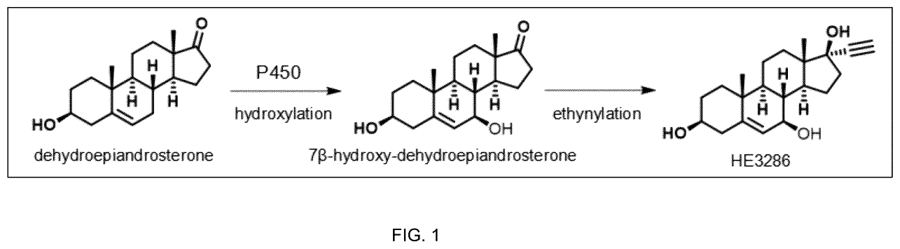

### DETAILED DESCRIPTION OF PREFERRED EMBODIMENTS

In the present specification and the subsequent claims, the term “comprise” and variations thereof (such as “comprises” or “comprising”) shall be construed to mean “including but not limited to,” and are not intended to exclude other additives, components, integers or steps. In the present specification and subsequent claims, the term “other” and its variants shall be construed to encompass not only those elements described in this patent, but also any readily substitutable methods, principles, or reagents that may be employed. When an element is described as “comprising” multiple components, steps, or conditions, it shall be construed to include: (i) any combination of such components, steps, or conditions; and (ii) embodiments where the element alternatively “consists of” or “consists essentially of” said components, steps, conditions, or combinations thereof.

To identify P450 enzymes capable of 7β-hydroxylation activity toward dehydroepiandrosterone, the inventors conducted systematic screening of existing P450 strains and, for the first time, discovered that the P450 BM3 mutant LG-23 exhibits exceptional 7β-hydroxylation activity on dehydroepiandrosterone.

All genetic elements (genes, expression cassettes, plasmids, or transformants) described herein can be prepared using conventional genetic engineering techniques.

The aforementioned transformants may comprise any microorganism suitable for expressing cytochrome P450 BM3 mutant, including both bacteria and fungi. Preferably, the microorganism is selected from the group consisting of Bacillus subtilis, Pichia pastoris, Saccharomyces cerevisiae, and Escherichia coli, with Escherichia coli being particularly preferred.

When serving as a biocatalyst, cytochrome P450 BM3 mutant may be utilized in either enzymatic or cellular forms. The enzymatic forms include free enzymes and immobilized enzymes, specifically encompassing purified enzymes, crude enzymes, fermented broth, or carrier-immobilized enzymes, among others. The cellular forms include viable cells, non-viable cells, immobilized cells, and the like.

As an alternative embodiment, microorganisms expressing cytochrome P450BM3 mutants can be utilized as biocatalysts for enzymatic reactions. The microorganisms may be used in the form of whole cells or their cell lysates. Whole-cell forms include both viable and non-viable cells, as microorganisms such as Bacillus subtilis, Pichia pastoris, Saccharomyces cerevisiae, or Escherichia coli-when no longer undergoing fermentation and proliferation but instead employed for enzymatic reactions-essentially function as naturally immobilized enzymes. Moreover, since both the reaction substrates and products are small-molecule compounds, they can readily traverse the biological barrier of the cell membrane. Therefore, there is no need for cell disruption or even purification, and the cells can be directly utilized as an enzyme preparation for catalytic reactions, which is economically advantageous.

More advantageously, many microbial cells inherently contain coenzymes such as NADP+ (nicotinamide adenine dinucleotide phosphate, Coenzyme II) or NAD+(nicotinamide adenine dinucleotide, Coenzyme 1), which can effectively facilitate redox reactions. This eliminates or reduces the need for additional supplementation of costly coenzymes in the enzymatic reaction system.

When employing cytochrome P450 BM3 mutants for the catalytic synthesis of steroid compounds, a cofactor regeneration system can be incorporated into the reaction system. As an optional embodiment, when using a combined catalytic system of cytochrome P450 BM3 mutants and glucose dehydrogenase (GDH), glucose may be added to the reaction mixture. Here, GDH catalyzes the oxidation of glucose while simultaneously reducing NADP+ (NAD+) to NADPH (NADH). The cytochrome P450 BM3 mutant then utilizes NADPH or NADH to catalyze the hydroxylation of the substrate. The optimal amounts of GDH and glucose to be added can be readily determined through straightforward experimental optimization.

As another optional embodiment, when using a combined catalytic system of cytochrome P450 BM3 mutants and alcohol dehydrogenase (ADH), isopropanol may be added to the reaction mixture. Here, ADH catalyzes the oxidation of isopropanol while simultaneously reducing NADP+ (NAD+) to NADPH (NADH). The cytochrome P450 BM3 mutant then utilizes NADPH or NADH to catalyze the hydroxylation of the substrate. The optimal amounts of ADH and isopropanol can be readily determined through routine experimental optimization.

Those skilled in the art will readily appreciate that the aforementioned glucose dehydrogenase and alcohol dehydrogenase may be provided either in the form of purified enzymes or as whole-cell preparations of expressing microorganisms.

In an optional embodiment, the cytochrome P450 BM3 mutant may be co-expressed with either glucose dehydrogenase or alcohol dehydrogenase within the same microbial strain, thereby eliminating the need for proportional addition of both enzymes or their expressing cells in the catalytic reaction system.

Furthermore, in addition to protection of the aforementioned mutants, this patent also discloses a novel technical approach employing a chemoenzymatic strategy for synthesizing steroid HE3286 using dehydroepiandrosterone as substrate. The method comprises the following steps:

The co-expressed or individually expressed P450 enzyme are resuspended in buffer solution, followed by addition of either isopropanol dehydrogenase or glucose dehydrogenase, dehydroepiandrosterone, cofactor NADP+, and isopropanol or glucose. The reaction is allowed to proceed to completion at 20-30° C. Ethyl acetate is then added to extract the reaction mixture, yielding an ethyl acetate extract. This extract is subsequently dried over anhydrous sodium sulfate, filtered under vacuum, and concentrated under reduced pressure to obtain crude 7β-hydroxy-dehydroepiandrosterone, which is further purified by recrystallization to afford pure 7β-hydroxy-dehydroepiandrosterone. Dissolve 7β-hydroxy-dehydroepiandrosterone in THF. Under ice-bath cooling and nitrogen protection, add dropwise a solution of ethynylmagnesium bromide in THF. Allow the reaction to proceed at 0-40° C. while monitoring the progress by TLC (dichloromethane:methanol=15:1) until complete substrate conversion is achieved. Upon reaction completion, quench the mixture with a saturated ammonium chloride solution and extract with an equal volume of ethyl acetate. Dry the resulting organic extract over anhydrous Na₂SO₄, filter, and concentrate under reduced pressure to remove the solvent. Add diisopropyl ether to the residue for slurrying, cool to induce crystallization, and isolate the product by suction filtration. After drying, the target compound HE3286 is obtained.

The P450 enzymes mentioned above include, but are not limited to, P450 BM3 mutants, and also encompass other P450 enzymes capable of hydroxylating the C7βposition of dehydroepiandrosterone. Among these, the selected P450 BM3 mutants represent the most optimal choice. The alkynylation reagents described herein include, but are not limited to, ethynylmagnesium bromide Grignard reagent, and also encompass other reagents capable of introducing an alkyne group at the C17th position of dehydroepiandrosterone, such as ethynylmagnesium chloride, acetylene, trimethylsilylacetylene, and calcium carbide. Among these, ethynylmagnesium bromide is the most optimal choice.

The substrates involved in the hydroxylation reaction described herein encompass not only dehydroepiandrosterone as a specific compound, but also include its precursors, key intermediates, and structurally analogous compounds. Representative examples include: androstenedione (CAS: 63-05-8), dehydroepiandrosterone acetate (CAS: 1239-31-2), androstenediol (CAS: 521-17-5), ethynyl androstenediol, and epiandrosterone analogs.

The 7β-hydroxylation reaction described herein is typically conducted in solvent systems. While water is the most preferred solvent, organic solvents—either alone or in combination with water—may be employed in certain cases. Suitable organic solvents include, but are not limited to, ethyl acetate, butyl acetate, 1-octanol, heptane, octane, methyl tert-butyl ether (MTBE), and toluene, as well as ionic liquids such as 1-ethyl-3-methylimidazolium tetrafluoroborate, 1-butyl-3-methylimidazolium tetrafluoroborate, and 1-butyl-3-methylimidazolium hexafluorophosphate. In preferred embodiments, aqueous solvent systems are utilized, including water and aqueous cosolvent systems. The solvent system preferably contains more than 50%, 75%, 90%, 95%, or 98% water by volume, and in one particular embodiment consists of 100% water.

The hydroxyl-protecting reagents described herein include, but are not limited to, TBDMSCI, and also encompass other silylating reagents capable of protecting hydroxyl groups, such as TBDMSOTf, TMSCI, TESCI, TBDPSCI, and TIPSCI. Among these, TBDMSCI represents the most optimal choice.

In this document, the addition amounts, contents, and concentrations of various substances are specified. Unless otherwise indicated, all percentage values mentioned refer to mass percentage (weight percent, wt %).

### Materials and Methods.

All gene synthesis, primer synthesis, and sequencing in the examples were performed by Sangon Biotech (Shanghai) Co., Ltd.

The molecular biology experiments in the Examples included plasmid construction, restriction digestion, ligation, competent cell preparation, transformation, culture medium preparation, etc., primarily performed according to Molecular Cloning: A Laboratory Manual (3rd Edition, J. Sambrook, D. W. Russell (eds.), Chinese translation by Huang Peitang et al., Science Press, Beijing, 2002). Experimental conditions could be determined through routine optimization when necessary.

The PCR amplification experiments were conducted according to the reaction conditions provided by the plasmid/DNA template supplier or the kit instructions. Optimization through routine testing was performed when necessary.

LB Medium Composition: 10 g/L tryptone, 5 g/L yeast extract, 10 g/L NaCl, pH adjusted to 7.2. LB Solid Medium:Additional 20 g/L agar.

TB Medium Composition: 24 g/L yeast extract, 12 g/L tryptone, 16.43 g/L K₂HPO₄.3H₂O, 2.31 g/L KH₂PO₄, 5 g/L glycerol, pH adjusted to 7.0-7.5. TB Solid Medium:Additional 20 g/L agar.

The dehydroepiandrosterone used in the Examples was purchased from Sigma-Aldrich.

All samples prepared in the Examples were analyzed using Shimadzu high-performance liquid chromatography (HPLC) systems (LC-2030 or LC-2030C). and dehydroepiandrosterone analysis conditions comprise: column, Agilent ZORBAX SB-C18 (250×4.6 mm); detection wavelength, 210 nm; mobile phase, acetonitrile and ultrapure water.

For descriptive convenience in the Examples, the same designation number may be shared across strain, plasmid, enzyme, and enzyme-encoding gene identifiers. Those skilled in the art will readily appreciate that a single identifier may refer to distinct biological forms depending on context.

The plasmids expressing cytochrome P450 BM3 mutants (e.g., pRSFDuet-LG-23) and those used for gene editing operations in the Examples were constructed and maintained by the research group of Professor Li Aitao at the College of Life Sciences, Hubei University. While these plasmids are available to any individual or organization for verifying the present invention, their use for other purposes-including development, commercialization, scientific research, and teaching-requires prior authorization from Hubei University.

The present disclosure is further elaborated below through specific examples. It should be understood that these examples are provided solely to illustrate the disclosure and shall not be construed as limiting the scope of the disclosure.

### Example 1

This example identified an enzyme capable of 7β-hydroxylation of dehydroepiandrosterone.

To obtain enzymes capable of 7β-hydroxylating dehydroepiandrosterone, we screened a library of P450 enzymes preserved in our laboratory. The preserved P450 enzyme-expressing E. coli strains from the laboratory library were streaked onto solid LB plates containing 50 μg/mL kanamycin and incubated overnight at 37° C. Single colonies were then picked and inoculated into 2 mL of liquid LB medium supplemented with 50 μg/mL kanamycin, followed by overnight shaking incubation at 37° C. Subsequently, 500 μL of the bacterial culture was transferred into a 100 mL Erlenmeyer flask containing 50 mL of TB medium and incubated at 37° C. with shaking at 220 rpm. When the OD600 of the culture reached 0.8, isopropyl β-D-1-thiogalactopyranoside (IPTG) was added at a final concentration of 0.2 mM to induce protein expression. Induction was carried out at 25° C. for 16-20 h. The culture was then centrifuged at 4,000 rpm for 10 min at 4° C. to harvest the cells, which were washed once with 100 mM potassium phosphate buffer (pH 8.0) and stored at −80° C.

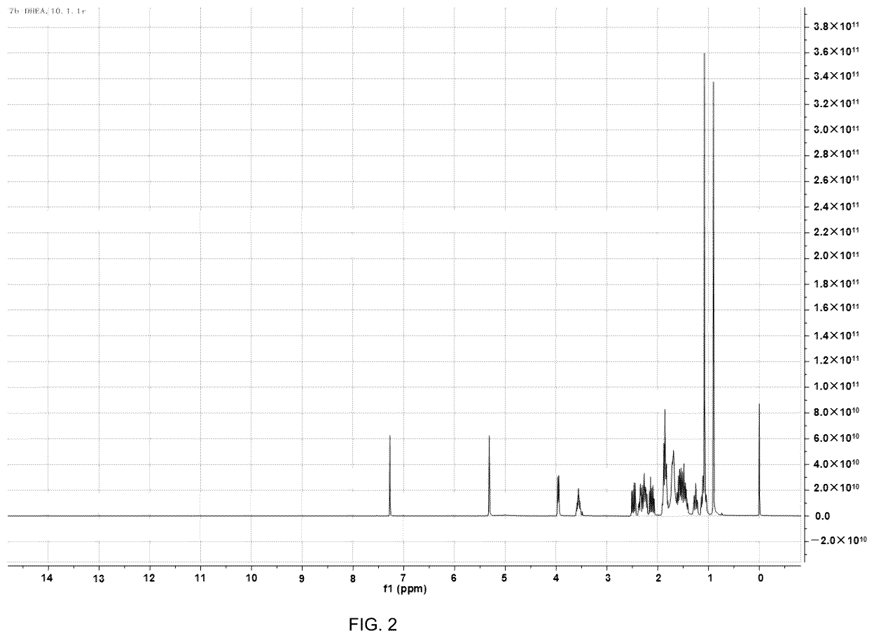

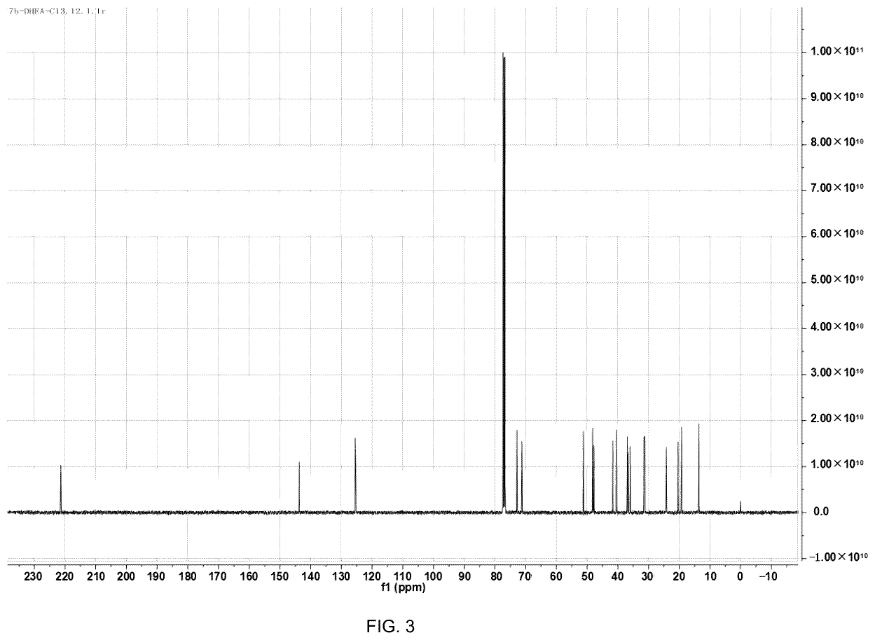

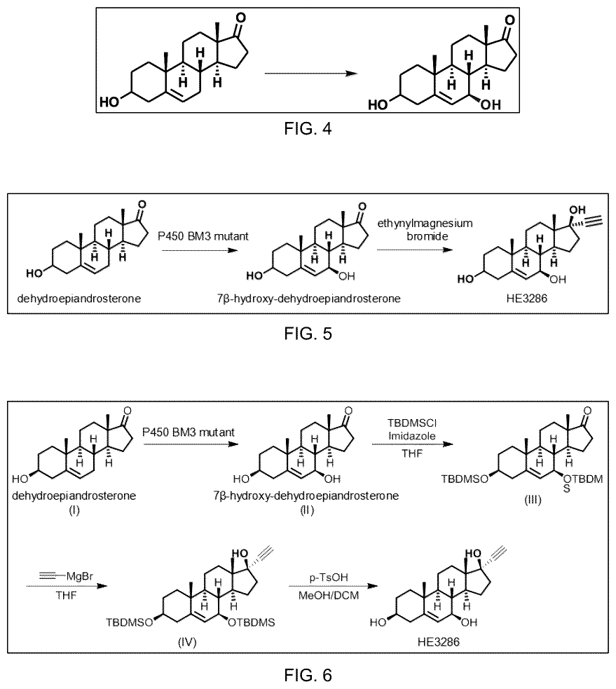

The cells were resuspended in 10 mL of 100 mM potassium phosphate buffer (pH 8.0) containing 5% (w/v) glucose, 5% (v/v) glycerol, 0.2 mM NADP+, and 10 U of GDH in a 50 mL centrifuge tube and immediately flash-frozen in liquid nitrogen. After thawing at room temperature in a water bath, 5 mL of the cell suspension was transferred to a 50 mL Erlenmeyer flask, supplemented with 1 g/L dehydroepiandrosterone, and incubated at 25° C. with shaking at 220 rpm for 24 h. At designated time intervals, samples were withdrawn, extracted with methanol, and centrifuged at high speed for 1 min. The supernatant was filtered through a 0.22 μm membrane into HPLC vials, and the conversion rate and product distribution were analyzed by HPLC. The obtained products were characterized, and the results demonstrated that the P450 BM3 mutant LG-23 exhibited the highest 7β-hydroxylation activity toward dehydroepiandrosterone. The nuclear magnetic resonance (NMR) spectra of the resulting product are shown in FIGS. 2 and 3. The reaction scheme for the C7β-hydroxylation of dehydroepiandrosterone catalyzed by the P450 BM3 mutant LG-23 is illustrated in FIG. 4. As shown in Table 1, the mutant LG-23 exhibited significantly higher catalytic activity compared to other P450 BM3 mutants.

TABLE 1

| | | product titer | Selectivity |
|---|---|---|---|
| P450 BM3 mutant | substrate | (g/L) | (%) |
| R19/F87A/A184I/A328G/T260G | DHEA | 0.3 | 93 |
| LG-23 | DHEA | 1 | 94 |

The P450 BM3 mutant LG-23 described in this study, along with its construction method, has been patented. The amino acid sequence of LG-23 is provided as SEQ ID NO:1, and its nucleotide sequence is listed as SEQ ID NO:2.

### Example 2

This example employed a chemoenzymatic method to synthesize steroid HE3286. Specifically, the mutant LG-23 was utilized for one-step biocatalysis of dehydroepiandrosterone to yield 7β-hydroxy-dehydroepiandrosterone, followed by a chemical alkynylation reaction to produce steroid HE3286. The reaction scheme is illustrated in FIG. 5.

### (1) Enzymatic Hydroxylation.

The whole cells of E. coli co-expressing the LG-23 mutant and isopropanol dehydrogenase were resuspended in 2 L of 100 mM potassium phosphate buffer (pH 8.0) with an OD600 of 40-60, then transferred to a 5 L bioreactor and stirred at 25° C. and 500 rpm. 2 g of dehydroepiandrosterone was added to the fermenter along with 40 mL of isopropanol. After complete addition, the mixture was stirred at 25° C. for 6-8 h until TLC analysis confirmed complete conversion of the starting material to 7β-hydroxy-dehydroepiandrosterone. The reaction mixture was then extracted four times with 2 L of ethyl acetate each time. All ethyl acetate extracts were combined, dried over anhydrous Na₂SO₄, filtered under vacuum, and concentrated under reduced pressure. The concentrated product was recrystallized to yield 1.9 g of 7β-hydroxy-dehydroepiandrosterone with a molar yield of 90%.

### (2) Alkynylation Reaction.

1 g of 7β-hydroxy-dehydroepiandrosterone was weighed and dissolved in THF. Under nitrogen protection and ice-bath cooling, ethynylmagnesium bromide (18 eq, 0.33 M in THF) was added dropwise. The reaction mixture was then warmed to 35° C. and stirred for 5 h. After TLC confirmed reaction completion, the mixture was quenched with saturated NH₄Cl solution and extracted with an equal volume of ethyl acetate. The organic phase was collected, and the extraction process was repeated three times. The combined organic phases were dried over anhydrous Na₂SO₄, filtered, and concentrated by rotary evaporation. The crude product was purified by trituration with isopropyl ether, followed by filtration and drying to yield 1.1 g of steroid HE3286 with a molar yield of 95%.

Steroid HE3286 was synthesized from dehydroepiandrosterone via a two-step reaction sequence with an overall yield of 86%.

### Example 3

This example employed a chemoenzymatic method to synthesize steroid HE3286. Initially, the mutant LG-23 catalyzed the one-step conversion of dehydroepiandrosterone to 7β-hydroxy-dehydroepiandrosterone. Subsequent steps involved protection of the 3rd and 7th position hydroxyl groups, followed by chemical alkynylation and deprotection to yield steroid HE3286. The reaction scheme is illustrated in FIG. 6.

### (1) Enzymatic Hydroxylation.

The whole cells of E. coli co-expressing the LG-23 mutant and isopropanol dehydrogenase were resuspended in 2 L of 100 mM potassium phosphate buffer (pH 8.0) with an OD600 of 40-60, then transferred to a 5 L bioreactor and stirred at 25° C. and 500 rpm. 2 g of dehydroepiandrosterone was added to the fermenter along with 40 mL of isopropanol. After complete addition, the mixture was stirred at 25° C. for 6-8 h until TLC analysis confirmed complete conversion of the starting material to 7β-hydroxy-dehydroepiandrosterone. The reaction mixture was then extracted four times with 2 L of ethyl acetate each time. All ethyl acetate extracts were combined, dried over anhydrous Na₂SO₄, filtered under vacuum, and concentrated under reduced pressure. The concentrated product was recrystallized to yield 1.9 g of 7β-hydroxy-dehydroepiandrosterone with a molar yield of 90%.

### (2) Hydroxyl Protection Reaction.

Compound II (7β-hydroxy-dehydroepiandrosterone, 1.80 g) was dissolved in THF (18 mL), followed by sequential addition of imidazole (1.94 g) and TBDMSCI (3.56 g). The reaction mixture was stirred at room temperature until completion, then quenched with water (18 mL) and extracted with ethyl acetate (3×20 mL). The organic layers were combined, dried over anhydrous Na₂SO₄, filtered, and concentrated under reduced pressure. The crude product was purified by trituration with n-hexane, filtered, and dried to afford 3.60 g of Compound Ill (3,7-hydroxyl-protected compound) in 99% molar yield.

### (3) Alkynylation Reaction.

Compound III (3.6 g) was dissolved in THE. Under nitrogen protection and ice-bath cooling, ethynylmagnesium bromide (18 eq, 0.33 M in THF) was added dropwise. The reaction mixture was then warmed to 35° C. and stirred for 5 h. After TLC confirmed complete consumption of the starting material, p-TsOH (2.47 g) was added, and the mixture was stirred under reflux. Upon reaction completion, the mixture was neutralized with saturated NaHCO₃ solution (20 mL) and extracted with DCM (3×20 mL). The combined organic extracts were dried over anhydrous Na₂SO₄, filtered, and concentrated under reduced pressure. The crude product was recrystallized from acetonitrile (28 mL), filtered, and dried to afford steroid HE3286 (2.2 g) in 95% molar yield.

Steroid HE3286 was synthesized from dehydroepiandrosterone via a three-step reaction sequence with an overall yield of 85%.

The above detailed embodiments thoroughly illustrate the implementation of the present invention; however, the invention is not limited to the specific details described in these embodiments. Within the scope of the claims and technical concept of the present invention, various simple modifications and alterations may be made to the technical solutions of the invention, all of which shall fall under the protection scope of the present invention.

What is claimed is:

1. A chemoenzymatic method for synthesizing steroid HE3286, comprising the following steps:
(1) converting dehydroepiandrosterone into 7β-hydroxy-dehydroepiandrosterone under the action of 7β-hydroxylase;
(2) alkynylating the carbonyl group at the C17th position of 7β-hydroxy-dehydroepiandrosterone to obtain steroid HE3286.

2. The chemoenzymatic method for synthesizing steroid HE3286 according to claim 1, wherein the 7β-hydroxylase is a cytochrome P450 enzyme, preferably a P450 BM3 mutant, and more preferably the P450 BM3 mutant LG-23, the amino acid sequence of the P450 BM3 mutant LG-23 is as shown in SEQ ID NO:1;
the alkynylation reaction specifically involves reacting 7β-hydroxy-dehydroepiandrosterone with ethynylmagnesium bromide, acetylene gas, or other ethynyl Grignard reagents.

3. The chemoenzymatic method for synthesizing steroid HE3286 according to claim 2, wherein step (1) specifically comprises:
reacting P450 BM3 mutant LG-23 with isopropanol dehydrogenase or glucose dehydrogenase, dehydroepiandrosterone, the cofactor NADP+, and isopropanol or glucose until completion;
extracting the reaction mixture with ethyl acetate to obtain a crude product of 7β-hydroxy-dehydroepiandrosterone, subsequently purifying the crude product via recrystallization to yield 7β-hydroxy-dehydroepiandrosterone as a pure product.

4. The chemoenzymatic method for synthesizing steroid HE3286 according to claim 1, wherein step (2) specifically comprises:
adding 7β-hydroxy-dehydroepiandrosterone to tetrahydrofuran, dropwise adding an ethynylmagnesium bromide/tetrahydrofuran solution under ice-bath cooling and nitrogen protection, reacting at 0-40° C., quenching the reaction mixture with saturated ammonium chloride solution upon completion, and extracting with ethyl acetate to obtain steroid HE3286.

5. The chemoenzymatic method for synthesizing steroid HE3286 according to claim 1, wherein step (2) comprises:
(21) dissolving 7β-hydroxy-dehydroepiandrosterone in an organic solvent, then sequentially adding an activator and TBDMSCI to obtain a 3,7-hydroxyl-protected compound;
(22) performing alkynylation at the C17th carbonyl group of the 3,7-hydroxyl-protected compound followed by deprotection to generate steroid HE3286.

6. The chemoenzymatic method for synthesizing steroid HE3286 according to claim 5, wherein step (22) specifically comprises:
adding a cosolvent to the 3,7-hydroxyl-protected compound and reacting with ethynylmagnesium bromide, acetylene gas, or other ethynyl Grignard reagents; after completion of the reaction, adding p-toluenesulfonic acid, followed by concentration under reduced pressure to obtain steroid HE3286.

7. The chemoenzymatic method for synthesizing steroid HE3286 according to claim 5, wherein in step (21):
the organic solvent is selected from tetrahydrofuran, acetonitrile, dichloromethane or N,N-dimethylformamide, preferably tetrahydrofuran;
the activator is selected from imidazole, pyridine, 4-dimethylaminopyridine, 2,6-lutidine, triethylamine, N,N-diisopropylethylamine or 1,8-diazabicyclo[5.4.0]undec-7-ene, preferably imidazole;
the molar ratio of TBDMSCI to 7β-hydroxy-dehydroepiandrosterone is 3-5:1, the molar ratio of imidazole to TBDMSCI is 1.2-1.5:1, and the reaction temperature does not exceed 50° C.

8. The chemoenzymatic method for synthesizing steroid HE3286 according to claim 6, wherein the cosolvent is selected from tetrahydrofuran, diethyl ether, isopropyl ether, methyl tert-butyl ether, ethylene glycol dimethyl ether, 2-methyltetrahydrofuran, or 1,4-dioxane;
the ethynyl Grignard reagent is ethynylmagnesium bromide; and the molar ratio of ethynylmagnesium bromide to the 3,7-hydroxyl-protected compound is 1.05-30:1.

9. The application of cytochrome P450 enzymes, vectors expressing the same, cells containing the same, compositions comprising the same, and immobilized enzyme products thereof in the production of steroid compounds, wherein the steroid compounds include steroid HE3286.

10. The application according to claim 9, wherein the P450 enzyme is a P450 BM3 mutant, preferably the P450 BM3 mutant LG-23, and the amino acid sequence of said P450 BM3 mutant LG-23 is as shown in SEQ ID NO:1.
